(19) United States
(12) Patent Application Publication
(10) Pub. No.: US 2007/0282372 A1
(43) Pub. Date: Dec. 6, 2007

(54) Guide forceps device for use with vertebral treatment device, system and methods of use

(76) Inventors: Joseph W. Yedlicka, Indianapolis, IN (US); Robert A. Till, Avon, IN (US); Nancy S. Yedlicka, Indianapolis, IN (US); Patricia M. Till, Avon, IN (US)

Correspondence Address: Mark S. Leonardo, Esq.;Brown Rudnick Berlack Israels LLP, One Financial Center, BOX IP, Boston, MA, 02111, US

(21) Appl. No.: 11/788,414

(22) Filed: Apr. 20, 2007

Related U.S. Application Data
(60) Provisional application No. 60/809,945, filed on Jun. 1, 2006.

(51) Int. Cl. A61B 17/00; A61B 17/58; A61F 5/00

(52) U.S. Cl. 606/205; 606/207; 606/86; 606/99

(57) ABSTRACT

A forceps is provided, which is adapted for treating a vertebral body. The forceps has a handle including an actuator pivotably connected therewith. A shaft extends from the handle with the proximal end being operatively engageable with the actuator. An elongated member extends through the shaft and has a proximal end and a distal end. The proximal end being fixed to the handle and the distal end includes opposing arms configured to grasp. The forceps may be configured for use with a bone drill. Portions of the forceps may be radiolucent. A vertebral treatment system and methods of use are also provided.

### CROSS REFERENCE TO RELATED APPLICATIONS

This patent application claims the benefit of U.S. Provisional Patent Application Ser. No. 60/809,945, filed on Jun. 1, 2006, the contents of which being incorporated herein by reference in its entirety.

### I. BACKGROUND OF THE INVENTION

A. Field of the Invention

The present disclosure relates to medical devices, components, and methods of use, such as vertebral treatment devices, fluid transfer devices, bone drills, bone drill assemblies, and bone cavity creation/enlargement devices, especially for treating vertebral body and sacral fractures, as well as lytic (destructive) tumor deposits in bone.

B. Background Information

Throughout the years and most recently in particular, various instruments have been developed for use in and for particular medical procedures and/or techniques requiring bone access and treatment. In some bone access procedures, it is necessary to create one or more holes in a bone or bone sections or to bore through the bone. Medical instruments known as bone drills have been developed for creating such holes and bores. Other instruments such as catheters, needles, guide needles, curettes and the like may then be introduced into the hole. On occasion, a cavity needs to be created or enlarged to facilitate treatment of a bone lesion. In some cases, artificial materials may be introduced into the vertebral body, such as cement into the cavity created in the bone, to treat vertebral or sacral fractures.

Medical procedures that require drilling into bone often require creating a cavity or enlarging a cavity in the bone. Examples of such medical procedures include vertebroplasty and/or vertebral augmentation procedures, sacroplasty, and osteoplasty.

Vertebroplasty is a procedure for treating vertebral compression fractures. Sacroplasty is a procedure for treating sacral fractures. Osteoplasty is a procedure for treating painful lytic (destructive) tumor deposits in bone. Osteoporosis is a common cause for vertebral compression fractures and sacral fractures, however, tumors involving the spine such as multiple myeloma and metastatic disease can also cause these fractures. A vertebral body compression fracture (VCF) is a fracture involving the vertebral body, which causes the vertebral body to be compressed or collapse. This can lead to shortening and tilting of the spinal column with a forward curvature. This forward curvature can lead to pulmonary and gastrointestinal complications. These fractures are extremely painful and debilitating with many of these patients needing wheelchairs for less painful ambulation; many of these patients are bed-ridden. Vertebroplasty is designed to stabilize VCFs and relieve pain. Vertebral height restoration and deformity reduction are also desired.

In vertebral augmentation and vertebroplasty, access needles are manually pushed or hammered into the fractured vertebral body using fluoroscopic (X-ray) guidance. Various instruments such as a curette may then be inserted through the access needles or tubes. At that point in vertebroplasty, an orthopedic bone filler/cement (e.g. PMMA) is instilled into the fractured bone. In vertebral augmentation, before the bone cement is instilled, balloon catheters are inserted through the access needles or tubes into the fractured vertebral body. The balloon catheters are inflated in an attempt to restore the compressed/collapsed vertebral body to its original height and also to create a cavity in the fractured bone. Following the balloon dilation, the balloons are removed and thicker bone cement instilled into the fractured vertebral body through the access needles or tubes. The cement hardens quickly for both procedures, providing strength and stability to the vertebra. The progress of both procedures is continually monitored in real time with fluoroscopic (X-ray) guidance.

In sacroplasty, access needles are manually pushed or hammered into the fractured sacrum using fluoroscopic (X-ray) or computed tomographic (CT) guidance. Various instruments such as curettes or balloons may then be inserted through the access needles. An orthopedic bone filler/cement (e.g. PMMA) is then instilled through the access needles/tubes into the fractured sacrum. This has been found to provide pain relief and stability. Procedural progress is continually monitored with CT and/or fluoroscopic guidance.

In osteoplasty, access needles are manually pushed or hammered into the lytic (destructive) bone tumor deposit using fluoroscopic (X-ray) or computed tomographic (CT) guidance. Various instruments such as curettes, balloons, or radiofrequency (RF) probes may be inserted through the access needles. An orthopedic bone filler/cement (e.g.) PMMA is then instilled through the access needles/tubes into the lytic deposit. This has been found to provide pain relief and stability. Procedural progress is continually monitored with CT and/or fluoroscopic guidance. It has been recognized, however, that filler, such as the cement for the treatment procedures described above, can flow out from the targeted bone through cracks in the bone and into undesirable structures and areas adjacent to the targeted bone such as spinal canal, neural foramina, and blood vessels. This disadvantageously can result in undesirable health risks to a patient.

In bone biopsies, needles are manually pushed or hammered into the bone in order to obtain a specimen. In bone infusions, needles are manually pushed or hammered into the bone in order to achieve bone access.

It has been recognized that it is desirable for a bone drill to place the access needles in the targeted bone using fluoroscopic (X-ray) or CT guidance. It has also been recognized that it is desirable for this bone drill to have a guide tube or access needle in conjunction with a drill bit, the guide tube surrounding the drill bit. The guide tube/access needle may then be used as a conduit into the targeted bone. This drill can also be used with various bits (such as a screwdriver) for various medical procedures. However, existing drills suffer from various design defects that make them unsuitable to be used with fluoroscopic (X-ray) or computed tomographic (CT) guidance for these procedures. It is often difficult to place needles or access devices into bone by manually pushing or hammering; also the currently used devices result in excessive radiation exposure to the operator (particularly the hands). Also, currently available bone curettes do not reliably create a cavity in the accessed bone and also result in excessive radiation exposure to the operator (particularly the hands).

Therefore, it would be desirable to overcome the disadvantages and drawbacks of the prior art with improved vertebral treatment devices and related methods of use. It is thus evident from the above that there is a need for an improved bone drill and related methods of use. It is evident that there is a need for improved drill bits to be used for these applications. It is evident from the above that there is a need for improved cavity creation/enlargement in the targeted bone. It is also evident that there is a need for operator radiation protection when using these devices. It would also be desirable if a vertebral treatment system is provided. Desirably, the vertebral treatment system has a largely radiolucent forceps that facilitates guidance and stability during a drilling procedure, particularly those performed using fluoroscopic (X-ray) guidance.

### II. SUMMARY OF THE INVENTION

Accordingly, an improved vertebral treatment device and related methods of use are provided for overcoming the disadvantages and drawbacks of the prior art. Desirably, the vertebral treatment device and methods disclosed include an improved bone drill and related methods of use. Desirably, a vertebral treatment system is provided that advantageously protects an operator from radiation to minimize the consequent health risks to a patient. Most desirably, the vertebral treatment system has a largely radiolucent forceps that facilitates guidance and stability during a drilling procedure. The forceps may have a radiation protection guard on its handle.

In one particular embodiment, in accordance with the principles of the present disclosure, a radiolucent forceps is provided, which is adapted for treating a vertebral body. The forceps may have a radiation protection guard on its handle. Radiation exposure to the operator's hand is decreased by increasing the distance between the patient/X-ray beam and the operator's hand and is also decreased by a radiation protection guard. The forceps has a handle including an actuator pivotably connected therewith. A shaft extends from the handle. A proximal end of the shaft operatively engages the actuator. An elongated member extends through the shaft and has a proximal end and a distal end. The proximal end is affixed to the handle and the distal end includes opposing arms configured to grasp.

Alternatively, the actuator may operatively engage the shaft to cause axial movement thereof relative to the elongated member. The shaft can be axially moveable between a retracted position, whereby the arms are in a substantially open position, and an extended position, whereby the arms are in a substantially closed position. The arms may define a cylindrical cavity in the closed position. The arms may be outwardly biased.

In another alternate embodiment, a vertebral treatment system is provided. The vertebral treatment system includes a bone drill configured for treating bone of a vertebral body. The bone drill includes a handle connected to a drive housing. The drive housing is connected to a head portion. The head portion includes a shaft extending therefrom. The shaft includes a drill bit and a sheath disposed about the drill bit. The shaft is coupled to a motor disposed with the drive housing via gearing such that the motor rotates the drill bit and the sheath.

Alternatively, the vertebral treatment system may further include a cavity drill having a body with a sheath extending therefrom and being mounted with the bone drill. The body supporting gearing that operatively couples the sheath to a motor of the bone drill for rotation of the sheath. The vertebral treatment system may further include a forceps, similar to those described herein. The head portion of the bone drill may include radio opaque markers disposed in a configuration to facilitate alignment of the shaft during a fluoroscopy procedure.

The various aspects of the present disclosure will be more apparent upon reading the following detailed description in conjunction with the accompanying drawings.

### III. BRIEF DESCRIPTION OF THE DRAWINGS

The above-mentioned and other features and objects of this disclosure, and the manner of attaining them, will become more apparent and the disclosure itself will be better understood by reference to the following description of embodiments of the disclosure taken in conjunction with the accompanying drawings, wherein:

FIG. 1 is a perspective view of a device employed in a vertebral treatment procedure constructed in accordance with the principles of the present disclosure;

FIG. 2 is a side perspective view of the device shown in FIG. 1;

FIG. 3 is a side perspective view, in cross section, of the device shown in FIG. 1;

FIG. 4 is an enlarged perspective view, in cutaway, of a distal end of the device shown in FIG. 1;

FIG. 5 is a perspective view of a bone drill constructed in accordance with the principles of the present disclosure; and

FIG. 6 is a perspective view of an alternate embodiment of a bone drill/cavity drill constructed in accordance with the principles of the present disclosure.

Like reference numerals indicate the similar parts throughout the figures.

### IV. DETAILED DESCRIPTION OF EMBODIMENTS OF THE INVENTION

The exemplary embodiments of the vertebral treatment device and methods of use disclosed are discussed in terms of medical apparatus and more particularly, in terms of vertebral treatment devices, bone drills, bone drill assemblies and bone cavity drills that can be employed for treating vertebral body and sacral fractures. The vertebral treatment devices may also be employed to treat lytic tumor deposits in bone. It is envisioned that the present disclosure may be employed with a range of applications including vertebroplasty and/or vertebral augmentation procedures, sacroplasty, osteoplasty, bone biopsies and infusions. It is further envisioned that the present disclosure may be used with other medical applications such as diagnosis, treatment and surgery.

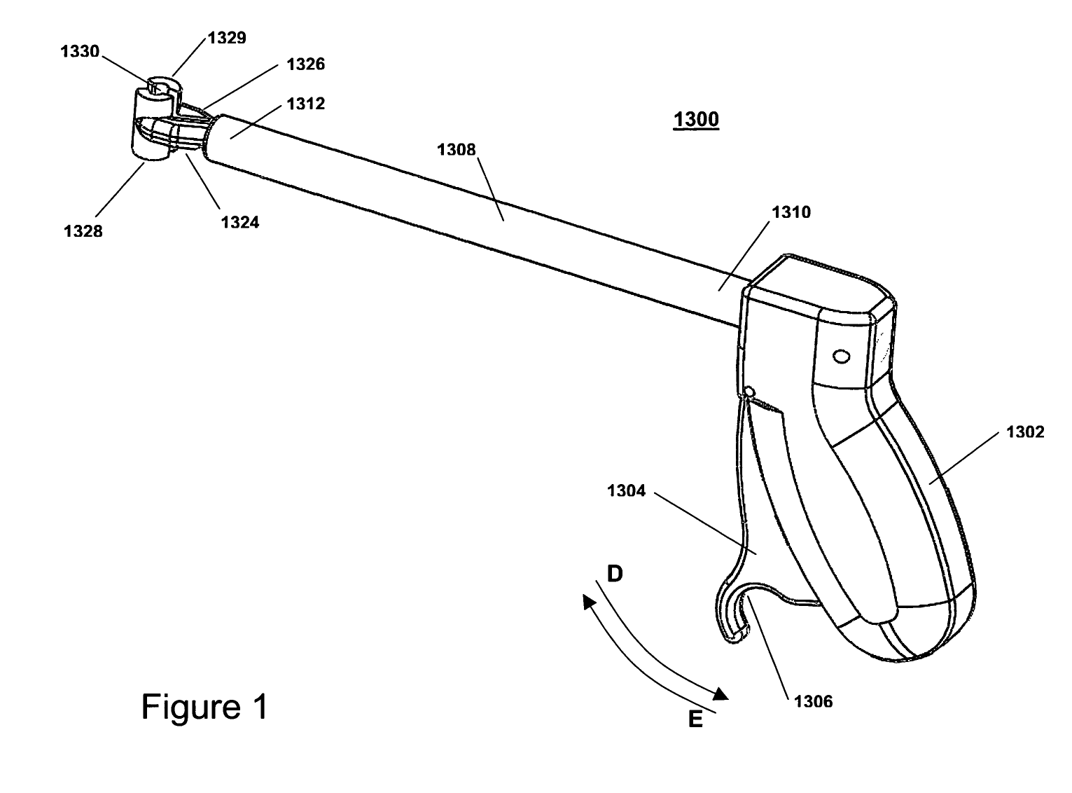

The following discussion includes a description of the vertebral treatment devices, related components and exemplary methods of operating the vertebral treatment devices in accordance with the principles of the present disclosure. Alternate embodiments are also disclosed. Reference will now be made in detail to the exemplary embodiments of the present disclosure, which are illustrated in the accompanying figures. Turning now to FIG. 1, there is illustrated a vertebral treatment device, such as, for example, a forceps 1300, in accordance with the principles of the present disclosure.

The components of forceps 1300 are fabricated from materials suitable for medical applications, such as, for example, polymerics and/or metals, depending on the particular application and/or preference. These materials may be radiolucent. Semi-rigid and rigid polymerics are contemplated for fabrication, as well as resilient materials, such as molded medical grade polyurethane, etc. It is contemplated that any motors, gearing, electronics and power components employed with forceps 1300 may be fabricated from those suitable for a medical application. Forceps 1300 may also include circuit boards, circuitry, processor components, etc. for computerized control. One skilled in the art, however, will realize that other materials and fabrication methods suitable for assembly and manufacture, in accordance with the present disclosure, also would be appropriate.

Detailed embodiments of the present disclosure are disclosed herein, however, it is to be understood that the described embodiments are merely exemplary of the disclosure, which may be embodied in various forms. Therefore, specific functional details disclosed herein are not to be interpreted as limiting, but merely as a basis for the claims and as a representative basis for teaching one skilled in the art to variously employ the present disclosure in virtually any appropriately detailed embodiment.

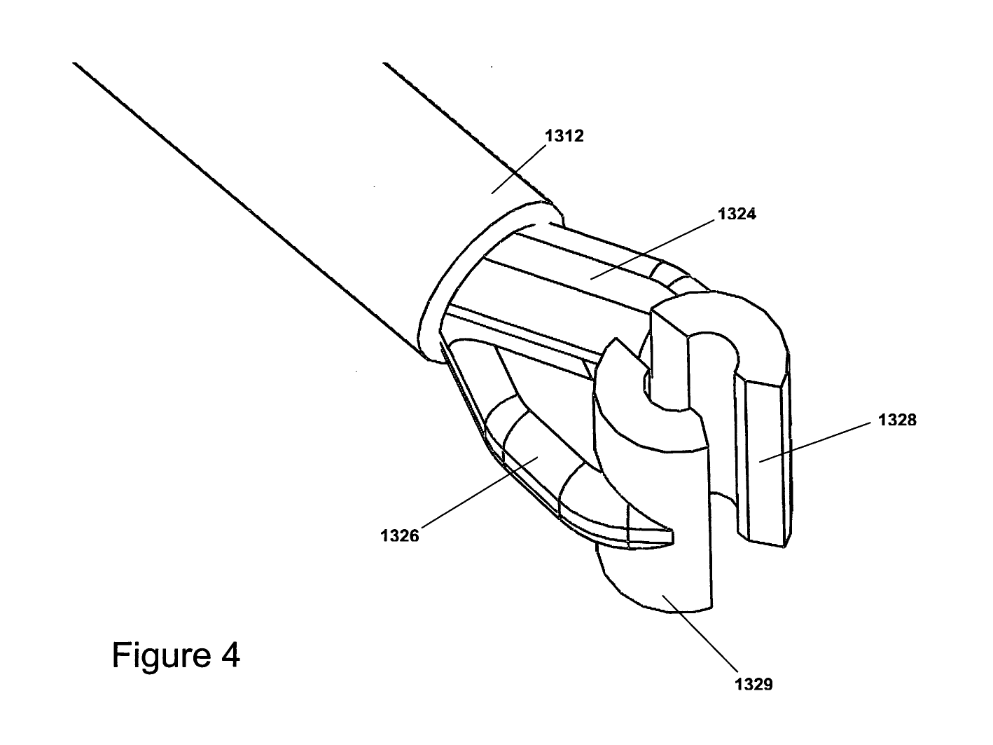

Referring to FIGS. 1-4, forceps 1300 is configured for use with a bone drill such as, for example, those bone drills described in co pending and commonly owned U.S. Utility Patent application Ser. No. ______, filed on Apr. 20, 2007 under Express Mail Label No. ER 550793142 US and U.S. Utility patent application Ser. No. ______, filed on Apr. 20, 2007 under Express Mail Label No. ER 550793139 US. Forceps 1300 is adapted to stabilize and guide a shaft, sheath and/or drill bit of a bone drill during treatment of a vertebral body, as will be described. Forceps 1300 is radiolucent such that at least a portion thereof is formed of a radiolucent material. It is contemplated that various components of forceps 1300 may be formed by radiolucent material and/or radiopaque material.

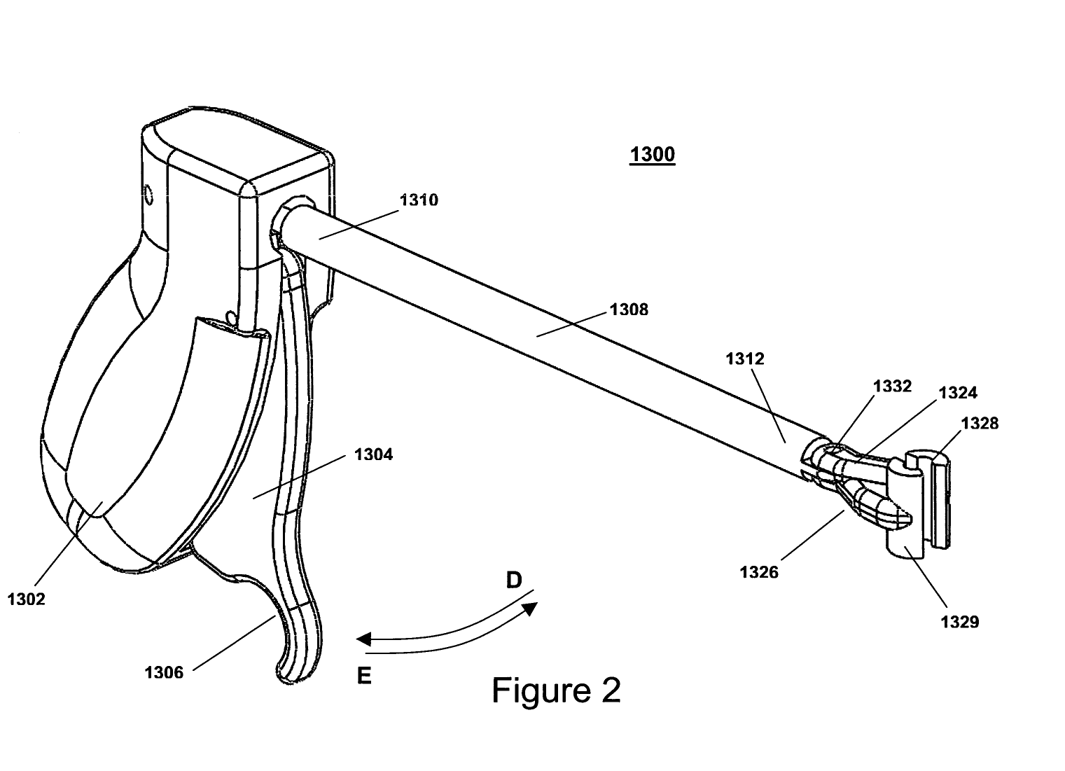

Forceps 1300 has a handle 1302 configured for grasping by a user's hand. A radiation protection guard may be attached to the handle. This guard may be fabricated from flexible or rigid radio-protective materials such as lead, tin, etc. The guard may be rotatable and/or removable. Handle 1302 has an actuator 1304, which is pivotably connected therewith. Actuator 1304 is manipulable inward towards handle 1302, in the direction shown by arrow D in FIGS. 1 and 2, by having a user's hand grasp or squeeze actuator 1304 with handle 1302. Actuator 1304 is manipulable outwardly away from handle 1302 by having a user's finger drive or push actuator 1304 at finger portion 1306, in the direction shown by arrow E.

A shaft 1308 extends from handle 1302 from a first end 1310 slidably mounted therewith to a second end 1312. Shaft 1308 has a tubular configuration to support an elongated member 1314. It is contemplated that shaft 1308 may be variously configured and dimensioned, for example, the length of shaft 1308 may be extended to protect the user from radiation. It is further contemplated that shaft 1308 may have various cross sectional configurations such as rectangular, elliptical, etc. It is envisioned that shaft 1308 may be fabricated from radiolucent material, as well as other components of forceps 1300, or alternatively, only shaft 1308 is fabricated from radiolucent material.

Elongated member 1314 extends through shaft 1308 and has a proximal end 1316 and a distal end 1318. Elongated member 1314 is fixed relative to handle 1302 at proximal end 1316. Distal end 1318 includes opposing arms 1324, 1326, which are configured to grasp. Shaft 1308 slides relative to handle 1302 and elongated member 1314. Proximal end 1310 is operatively engageable with actuator 1304. Actuator 1304 includes a tab 1320 configured for disposal within an opening 1322 defined in the proximal end 1310. It is envisioned that elongated member 1314 may be fabricated from radiolucent material, as well as other components of forceps 1300, or alternatively, only elongated member 1314 is fabricated from radiolucent material.

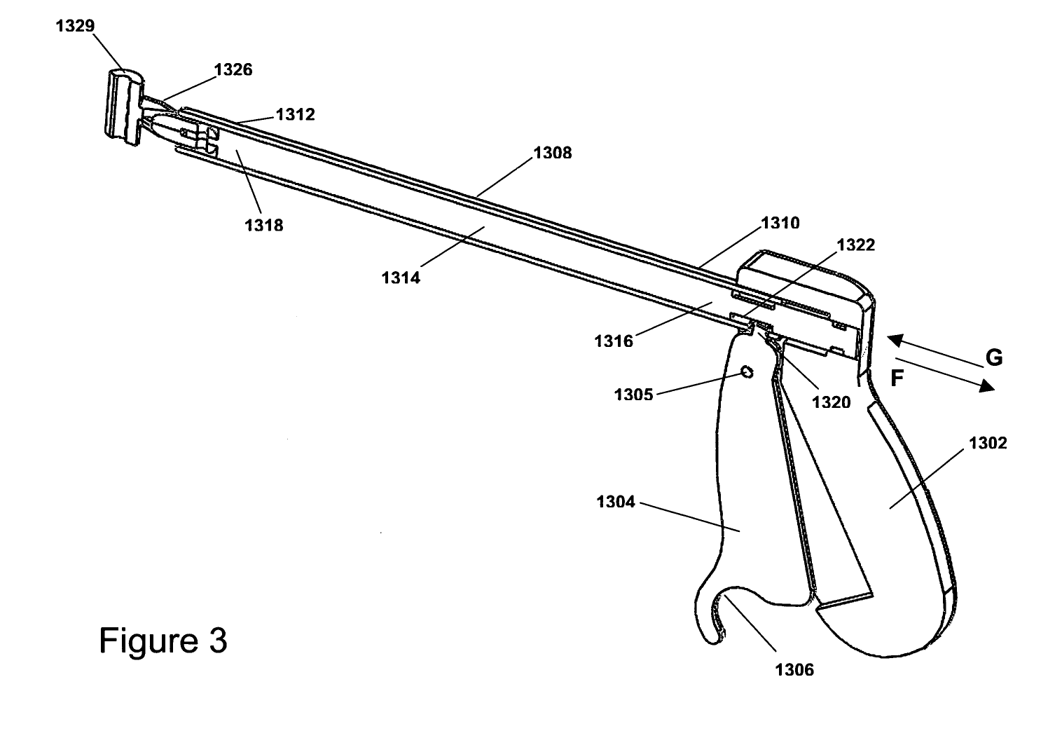

Tab 1320 engages a proximal end of opening 1322 to drive shaft 1308 in a proximal direction relative to elongated member 1314, in the direction shown by arrow F in FIG. 3. Tab 1320 engages a distal end of opening 1322 to drive shaft 1308 in a distal direction relative to elongated member 1314, in the direction shown by arrow G. A user manipulates actuator 1304 with handle 1302, as described to cause axial movement of shaft 1308 relative to elongated member 1314.

When shaft 1308 is forced distally (direction arrow G) it pushes on the angled sides of arms 1324, 1326 of jaws 1328, 1329 forcing them together thereby gripping whatever shaft is positioned within the jaws. A spring mounted within the cylindrical cavity of handle 1302 whose proximal side pushes against the inside wall of said cavity and whose distal end presses against the proximal end 1310 of shaft 1308 biases shaft 1308 distally applying a slight closing pressure on arms 1324, 1326. This allows the jaws to snap open and then closed around a shaft that is forced into the distal ends of jaws 1328, 1329. It is envisioned that arms 1324, 1326 and/or jaws 1328, 1329 may be fabricated from radiolucent material, as well as other components of forceps 1300, or alternatively, only arms 1324, 1326 and/or jaws 1328, 1329 are fabricated from radiolucent material.

Shaft 1308 is axially moveable between a retracted position whereby arms 1324, 1326, which include jaws 1328, 1329 and define a cylindrical cavity 1330, are in a substantially open position, and an extended position whereby arms 1324, 1326 are in a substantially closed position.

Opposing arms 1324, 1326 are pivotably connected at distal end 1318 by hinge 1332. Jaws 1328, 1329 may be biased outwardly by a resilient hinge connection of arms 1324, 1326 at hinge 1332. It is contemplated that arms 1324, 1326 may be biased via a spring, elastics, etc. It is further contemplated that arms 1324, 1326 may be manually moveable or moveable through mechanical advantage via the engagement of the components of forceps 1300.

In the retracted position, shaft 1308 is in its proximal most position relative to elongated member 1314. Actuator 1304 is in its forward most position with tabs 1320 engaging the proximal end of opening 1322. Arms 1324, 1326 are extended from shaft 1308 and jaws 1328, 1329 are in the open position.

To grasp an object, such as a shaft, sheath, drill bit, etc., the user grasps handle 1302 and engages finger portion 1306 to drive it forward, in the direction shown by arrow E. This causes tab 1320 to drive shaft 1308 rearwardly such that arms 1324, 1326 are extended from shaft 1308 in an open position allowing the jaws to slide over the object that is to be grasped. As the user then squeezes actuator 1304 and handle 1302, tab 1320 moves axially to engage the distal end of opening 1322. Shaft 1308 is driven forward to the extended position. This causes arms 1324, 1326 to be forced together into the closed position. The inner wall of the shaft 1308 engages arms 1324, 1326, overcoming their outward bias, and drawing jaws 1328, 1329 together to the closed position to grasp an object. Cylindrical cavity 1330 is configured to fit with the object being grasped. This advantageous configuration of forceps 1300 facilitates guidance and stabilizes various instruments that may be employed during a vertebral treatment procedure. It is envisioned that jaws 1328, 1329 may define a cylindrical cavity having alternate configurations such as elliptical, transverse, polygonal, etc.

In another particular embodiment, in accordance with the principles of the present disclosure, a vertebral treatment system is provided. The vertebral treatment system includes components such as a bone drill, forceps and a cavity drill for treating fractured bone of a vertebral body and/or a sacral body. It is envisioned that the vertebral treatment system may include one or all of the components discussed herein. It is further envisioned that the vertebral treatment system may include other components applicable to a vertebral treatment procedure and in accordance with the present disclosure.

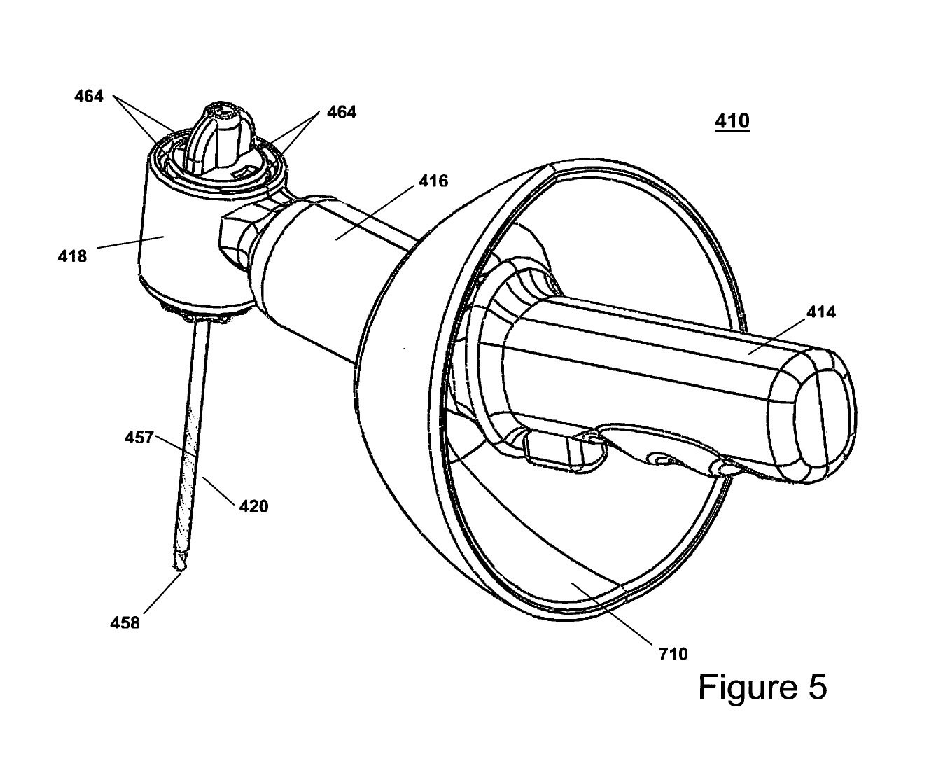

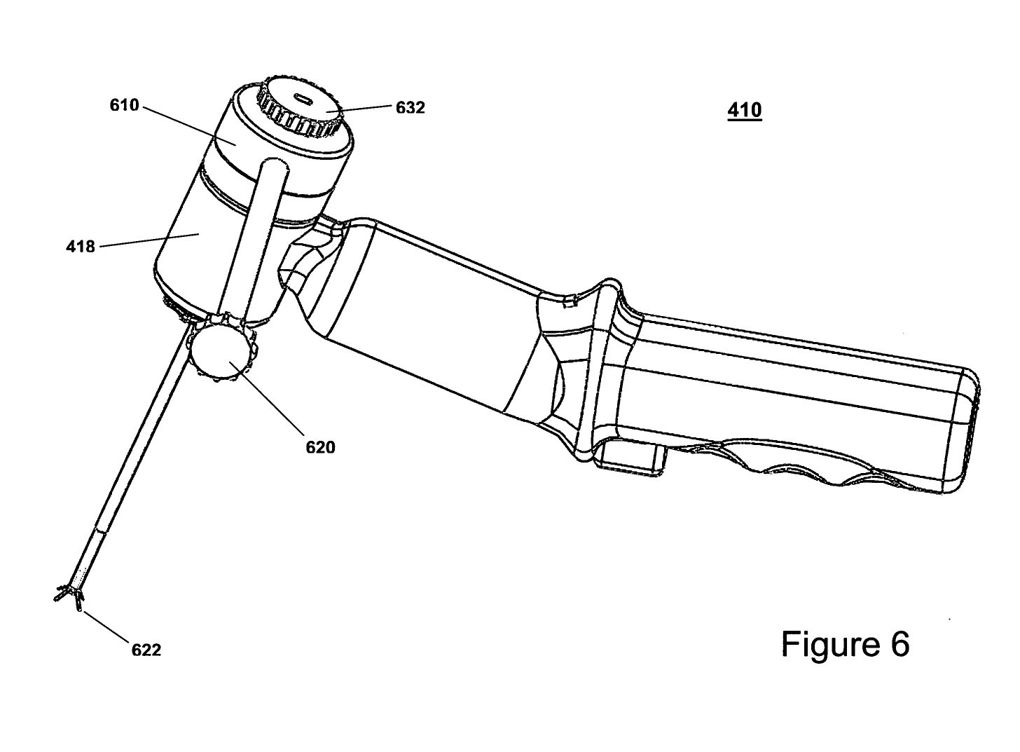

The vertebral treatment system employs, for example, a bone drill 410, as shown in FIG. 5, and a cavity drill 610, as shown in FIG. 6. See, for example, the description of the bone drills and the cavity drills disclosed in co pending and commonly owned U.S. application Ser. No. ______, filed on Apr. 20, 2007 under Express Mail Label No. ER 550793142 US and U.S. application Ser. No. ______, filed on Apr. 20, 2007 under Express Mail Label No. ER 550793139 US, the entire contents of these disclosures being incorporated by reference herein. It is envisioned that the vertebral treatment system may employ alternative components. Other uses of the described components of the vertebral treatment system are also contemplated.

In operation of the vertebral treatment system, bone drill 410 is employed with a method for treating fractured bone of a vertebral body or a sacral body. The components of bone drill 410 are fabricated, properly sterilized and otherwise prepared for use. Bone drill 410 is provided with handle portion 414, drive portion 416 and head portion 418 in a configuration that provides a safe distance between a physician and radiation emitted during the procedure.

Head portion 418 includes radiopaque markers 464 disposed in a configuration to facilitate alignment of sheath 457 with bone of the vertebral body. During fluoroscopy, an area is exposed to radiation, which includes bone drill 410 and the bone of the vertebral body. The exposure of radiation to bone drill 410 and radiopaque markers 464 allows the user to identify the location of sheath 457 and drill bit 458 relative to the targeted bone. This configuration facilitates alignment, via radiopaque markers 464, for cutting the bone while protecting the user by maintaining the offset angular orientation of bone drill 410. A guard 710 may also be used during the procedure.

Forceps 1300 is provided to stabilize and guide bone drill 410 during drilling of bone of the vertebral body. Forceps 1300 includes radiolucent arms 1324, 1326 having jaws 1328, 1329. This allows the user to see drill bit 458 and sheath 457, which are radiopaque, and the underlying bone structures. This configuration facilitates guidance for drilling and protects the user from radiation by maintaining the hands of the user a safe distance therefrom.

Arms 1324, 1326 are moveable between a closed position and an open position, as discussed above. When jaws 1328, 1329 are in the open position, sheath 457 is free to rotate. To grasp sheath 457 for guidance and stabilization of bone drill 410 during the vertebral drilling procedure, the user grasps handle 1302 and squeezes on actuator 1304. Shaft 1308 moves to the extended position and jaws 1328, 1329 move to the closed position to grasp sheath 457. Cylindrical cavity 1330 is configured to snugly fit and snap onto sheath 457. Sheath 457 is firmly held in position by forceps 1300, which advantageously operates as a drill guide.

Drill bit 458 engages the bone and rotates via motor 498 to bore a cavity in the bone. Sheath 457 is driven into engagement with the bone to further define the cavity in the bone. After drill bit 458 has reached a desired depth within the targeted bone, according to the requirements of a particular procedure, actuator 1304 of forceps 1300 can release jaws 1328, 1329 from sheath 457. Sheath 457 is free to rotate. If desired, forceps 1300 may be removed from sheath 457.

Cavity drill 610, which is an alternate embodiment of bone drill 410, is provided for enlarging and/or further defining the cavity bored in the bone by bone drill 410. Cavity drill 610 includes a knob 620, which is manipulated for rotation to drive a bone curette 622, which reams the targeted bone and cavity. Cavity drill 610 also includes a knob 632, which is manipulated for rotation to cause relative axial translation of bone curette 622. Knobs 620, 632 are rotated, in cooperation to ream the targeted bone area and further define the targeted bone cavity. It is contemplated that cavity drill 610 may include radiopaque markers to facilitate alignment thereof with the targeted bone. A radiation protection guard 710 may be fabricated from flexible or rigid radio-protective materials such as lead, tin, etc.; the guard may be rotatable and/or removable.

After the cavity is created in the targeted vertebral bone, according to the requirements for the particular fracture and treatment procedure, the targeted vertebral body or sacral body is treated. See, for example, the description of the methods of use described in co pending and commonly owned U.S. application Ser. No. ______, filed on Apr. 20, 2007 under Express Mail Label No. ER 550793142 US and U.S. application Ser. No. ______, filed on Apr. 20, 2007 under Express Mail Label No. ER 550793139 US. It is contemplated that one or a plurality of cavities may be created to allow for access tubing, cannulas, etc. in the targeted area. It is further contemplated that balloon catheters, etc., may be inserted through the access tubing, cannulas, etc. into the targeted fractured vertebral body. It is envisioned that the access tubing, cannulas, etc. may be fabricated from radiolucent material and/or radiopaque material. It is contemplated that bone cement may be instilled through the access tubing, cannulas, etc. into the targeted bone.

While the invention has been illustrated and described in detail in the drawings and foregoing description, the same is to be considered as illustrative and not restrictive in character, it being understood that embodiments have been shown and described and that all changes and modifications that come within the spirit of this invention are desired to be protected.

What is claimed is:

1. A radiolucent forceps adapted for treating bone, the forceps comprising:
a handle including an actuator pivotably connected therewith;
a shaft extending from the handle, a proximal end of the shaft being operatively engageable with the actuator; and
an elongated member extending through the shaft and having a proximal end and a distal end, the proximal end being affixed to the handle and the distal end including opposing arms configured to grasp.

2. A forceps as recited in claim 1, wherein the actuator operatively engages the shaft to cause axial movement thereof relative to the elongated member.

3. A forceps as recited in claim 2, wherein the shaft is axially moveable between a retracted position, whereby the arms are in a substantially open position, and an extended position, whereby the arms are in a substantially closed position.

4. A forceps as recited in claim 3, wherein the arms define a cylindrical cavity in the closed position.

5. A forceps as recited in claim 1, wherein the arms are outwardly biased.

6. A forceps as recited in claim 1, wherein the arms are outwardly biased via a spring.

7. A forceps as recited in claim 1, wherein the elongated member and the shaft are fabricated from a radiolucent material.

8. A forceps as recited in claim 1, wherein the arms are fabricated from a radiolucent material.

9. A forceps as recited in claim 1, wherein the arms are configured to support a bone drill shaft.

10. A vertebral treatment system comprising:
a bone drill configured for treating bone of a vertebral body, the bone drill including a handle moveably connected to a drive housing, the drive housing being moveably connected to a head portion, the head portion including a shaft extending therefrom, the shaft including a drill bit and a sheath disposed about the drill bit, the shaft being coupled to a motor disposed with the drive housing via gearing such that the motor rotates the drill bit and the sheath, wherein the head portion is disposed at an angular orientation relative to the handle.

11. A vertebral treatment system as recited in claim 10, further comprising a cavity drill including a body having a sheath extending therefrom and being mounted with the bone drill, the body supporting gearing that operatively couples the sheath to a motor of the bone drill for rotation of the sheath.

12. A vertebral treatment system as recited in claim 10, further comprising a forceps including a handle including an actuator pivotably connected therewith, a shaft extending from the handle with its proximal end being operatively engageable with the actuator, and an elongated member extending through the shaft and having a proximal end and a distal end, the proximal end being affixed to the handle and the distal end including opposing arms configured to grasp.

13. A vertebral treatment system as recited in claim 10, wherein the head portion includes radio opaque markers disposed in a configuration to facilitate alignment of the shaft during a fluoroscopy procedure.

14. A vertebral treatment system as recited in claim 10, wherein at least a portion of the bone drill is radiolucent.

15. A vertebral treatment system as recited in claim 11, wherein the cavity drill includes radio opaque markers disposed in a configuration to facilitate alignment during a fluoroscopy procedure.

16. A vertebral treatment system as recited in claim 11, wherein at least a portion of the cavity drill is radiolucent.

17. A vertebral treatment system as recited in claim 12, wherein at least a portion of the elongated member of the forceps is radiolucent.

18. A vertebral treatment system as recited in claim 12, wherein the jaws are configured to grasp the shaft of the bone drill.

19. A largely radiolucent forceps device designed to provide radiation protection for the operator's hand by increasing the distance between the patient/X-ray beam and the operator's hand.

20. The forceps as recited in claim 19, wherein the forceps may have a radiation protection guard on its handle; the guard may be rotatable and/or removable.

21. A vertebral treatment system adapted for treating bone lesions including vertebral and sacral fractures, bone tumors, for performing bone biopsies/infusions, and for facilitating other medical procedures requiring fluoroscopic guidance comprising:
a bone drill configured for treating bone of a vertebral body, the bone drill including a handle moveably connected to a drive housing, the drive housing being moveably connected to a head portion, the head portion including a shaft extending therefrom, the shaft including a drill bit and a sheath disposed about the drill bit, the shaft being coupled to a motor disposed with the drive housing via gearing such that the motor rotates the drill bit and the sheath, wherein the head portion is disposed at an angular orientation relative to the handle;
a cavity drill including a body having a sheath extending therefrom and being mounted with the bone drill, the body supporting gearing that operatively couples the sheath to a motor of the bone drill for rotation of the sheath; and
a forceps including a handle including an actuator pivotably connected therewith, a shaft extending from the handle with its proximal end being operatively engageable with the actuator, and an elongated member extending through the shaft and having a proximal end and a distal end, the proximal end being affixed to the handle and the distal end including opposing arms configured to grasp.

22. A forceps as recited in claim 1, wherein the forceps is configured for use with X-ray (fluoroscopic) guidance.
